(19) United States
(12) Patent Application Publication
(10) Pub. No.: US 2025/0325396 A1
(43) Pub. Date: Oct. 23, 2025

(54) SYSTEM FOR APPLYING A URINAL CONDOM

(71) Applicant: Samuel EHRENSPERGER, Düsseldorf (DE)

(72) Inventor: Samuel EHRENSPERGER, Düsseldorf (DE)

(73) Assignee: URISAN GMBH, Düsseldorf (DE)

(21) Appl. No.: 18/868,418

(22) PCT Filed: May 24, 2023
(86) PCT No.: PCT/IB2023/055332

§ 371 Date: Nov. 22, 2024

(30) Foreign Application Priority Data
May 25, 2022 (EP) 22000138.2

(51) Int. Cl. A61F 5/453; A61M 1/00

(52) U.S. Cl. CPC A61F 5/453; A61M 1/80

(57) ABSTRACT

A system for applying a urinal condom which enables the application of a urinal condom for patients. The system ensures a good fit of the urinal condom and simplifies application. The system includes a receiving bell, a vacuum pump and a connecting unit for fluidically connecting the receiving bell to the vacuum pump. The connecting unit has a tubular channel, to which the receiving bell is fluidically connected. The tubular channel being designed on the receiving bell side as a receptacle for a hose attachment of the urinal condom. The connecting unit has a surface on the side to be connected to the vacuum pump, which is congruent with a surface formed on the vacuum pump. At least one of the two surfaces have an uneven structure, and the vacuum pump and the connecting unit are fluidically connected via the two superimposed surfaces.

The present invention relates to a system for applying a urinal condom for use as intended. Incontinence, also known as bladder weakness, is a disability that is also increasing as societies age. Uncontrolled leakage of fluid is either absorbed using absorbent aids, such as diapers and the like, or drained away.

Catheters are used for drainage, which are either inserted directly into the bladder through the abdominal wall, which can often lead to infections, or are pushed into the bladder through the urethra of the penis and can lead to injuries to the urethra.

An alternative are so-called urinal condoms, which can be connected to a drainage tube, collection bags and the like.

Urinary condoms are a preferred alternative for the treatment of male urinary incontinence. One disadvantage is that it can be difficult to put on a urinary condom. If dexterity is limited, this must be done by strangers, nursing staff or similar. For a variety of reasons, the limb receiving the condom often proves to be less receptive. This often leads to leaks or folds, poor fit and the like. Patients with a retracted penis or impotence have not been able to put on and use urinary condoms at all.

A device for attaching a urinal is known from the prior art from CH 672 591 A5. The device makes it possible to attach a urinal in a simplified manner by placing the penis cuff of the urinal over the edge of the opening of a cup-shaped vessel of this device and generating a vacuum inside this vessel.

This causes the cuff, which is closed at the outlet end, to inflate and allows easy access to the inside.

EP 3346962 B1 discloses a generic device for applying a urinal condom for the intended use, which generally considerably simplifies the application of a urinal condom. The known device comprises a receiving bell, a vacuum pump and a connection unit for fluidically connecting the receiving bell to the vacuum pump. A cup-shaped part of the device into which a urinal condom can be inserted is referred to as a receiving cup. Urinal condoms are usually rolled up. They have a tube-like attachment at the tip. The receiving bell is designed in such a way that it offers a possibility for inserting the condom for the respective size of a urinal condom, which lies against the bell-shaped inner surface of the receiving device. The tube-like attachment protrudes through an opening at the corresponding point on the receiving bell. The connecting unit has a tubular channel so that when the receiving bell is connected to the connecting unit, the tubular extension of an inserted condom protrudes into the channel. The connection unit is in turn connected to a device for generating a vacuum. For the intended use, a urinal condom is inserted into the receptacle bell in such a way that the tube attachment protrudes through a lower opening of the receptacle bell into the tubular channel of the connection unit and the condom ring comes to rest against the free upper edge of the receptacle bell. The condom area in between rests against the bell-shaped inner area of the receiving bell. The device prepared in this way is now brought up to a penis as intended and the penis is sucked into the area of the urinal condom in contact with the receiving bell due to the negative pressure generated by the operation of the vacuum pump. By switching off the vacuum pump, the device can now be removed so that the condom remains on the penis. It can now simply be unrolled as intended and is therefore optimally attached. It is also possible to remove the device while the vacuum pump is running, which may be adjustable, so that in certain cases a wrinkle-free unrolling of the urinal condom is also supported.

In practical use, it has been found that there are various problems in the area between the connection unit on the one hand and the device for generating the vacuum on the other. Either the connection is not tight enough so that the vacuum generated is not sufficient to suck the condom completely into the bell, or the connection is so tight that the pump can no longer be detached from the connection unit because the two have become stuck together.

Based on the state of the art described above, the present invention is based on the task of improving a system for applying a urinal condom of the type according to the present invention with regard to trouble-free and safe use, so that it ensures a flow-tight connection between the device for generating a vacuum on the one hand and the connection unit on the other, without hindering the detachability of the two units from each other.

A device with the features of patent claim 1 is proposed as a technical solution. Further advantages and features are shown in the sub-claims.

The system according to the invention comprises a vacuum pump on the one hand and a connection unit on the other. Both are placed against each other in such a way that they lie against each other with congruent surfaces. At least one of the two surfaces has an uneven structure. On the side of the vacuum pump, a suction opening opens into the surface provided for the connection. On the side of the connection unit, a suction channel opens out, which is connected to the suction bell or the holding bell. When the vacuum pump and the connection unit are put together, the vacuum generated by the vacuum pump can act through the connection unit to the suction bell. At least one of the two surfaces is provided with an uneven structure to prevent the two surfaces lying on top of each other from sticking to each other.

According to a further advantageous proposal of the invention, the two surfaces lying on top of each other are sealed at the edges. This can be achieved in different ways. For example, a circumferential sealing ring between the two surfaces can provide a seal. According to an advantageous proposal of the invention, one of the surfaces is surrounded by an annular sealing edge into which the respective other device can be inserted in order to place its surface intended for connection onto the other surface. In addition, the sealed edge can be sealed off from the housing by a further sealing ring.

For its part, the device comprises a receiving bell, a vacuum pump and a connection unit for fluidically connecting the receiving bell to the vacuum pump. A cup-shaped part of the device into which a urinal condom can be inserted is referred to as a receiving bell. Urinal condoms are usually rolled up. They have a tube-like attachment at the tip. The receiving bell is designed in such a way that it provides a way of inserting the condom for the respective size of urinal condom, which fits against the bell-shaped inner surface of the receiving device. The tube-like attachment protrudes through an opening at the corresponding point on the receiving bell.

A connection unit has a tubular channel, so that when the receiving bell is connected to the connection unit, the tube attachment of an inserted condom protrudes into the channel. The connection unit is in turn connected to a device for generating a vacuum. This can be a pump ball or pump scissors, which are operated manually, or an electric pump for mains or battery operation. In the simplest case, the connection unit is simply a tube that is attached to the bell jar in such a way that it can accommodate the tube attachment of an inserted condom and connects the vacuum pump to the interior of the bell jar in terms of flow. In order to optimize the application, the inner contour of the receiving bell is designed in such a way that it can accommodate a urinal condom of a specified size with an exact fit. This prevents wrinkles from forming and supports optimal application. At the intended outer upper edge, i.e. the free upper edge which is not connected to a connecting unit, the receiving bell has a circumferential groove according to an advantageous proposal of the invention. This serves the purpose of receiving the condom ring, which results from the rolling up of the urinal condom, when the urinal condom is inserted into the receiving bell. In the event that no groove is formed, the invention also provides for the condom ring to be slipped over the edges of the receiving bell or, for example, to be fastened with a clamping ring.

In an advantageous way, the receiving bell is cylindrical on the outside in order to provide an appropriate feel for good handling. In order to be usable for different urinal condoms, the receiving bell can be interchangeably connected to the connection unit. In this way, the connection unit can be connected to receiving bells of different sizes and designs.

The entire device can be provided with an integrating housing in which, for example, a battery-operated electric pump, a connecting channel and a holding bell are accommodated, whereby the holding bell can be arranged in a detachable housing part.

For the intended use, a urinal condom is inserted into the receiving bell in such a way that the tube attachment protrudes through a lower opening of the receiving bell into the tubular channel of the connection unit and the condom ring comes to rest against the free upper edge of the receiving bell. The condom area in between rests against the bell-shaped inner area of the receiving bell. The device prepared in this way is now brought up to a penis as intended and the penis is sucked into the area of the urinal condom lying against the receiving bell due to the negative pressure generated by the operation of the vacuum pump.

Sizes, softness and the like are compensated for by simple suction so that the urinal condom is optimally positioned. By switching off the vacuum pump, the device can now be removed so that the condom remains on the member. It can now simply be unrolled as intended and is therefore optimally attached. It is also possible to remove the device while the vacuum pump is running, which may be adjustable, so that in certain cases a wrinkle-free unrolling of the urinal condom is also supported.

The invention provides a device that is easy to manufacture and, above all, easy to use, with which the application of a urinal condom for its intended use is considerably simplified and optimized.

Further advantages and features are shown in the following description based on the figures. They show:

FIG. 1 a schematic representation of a typical urinal condom;

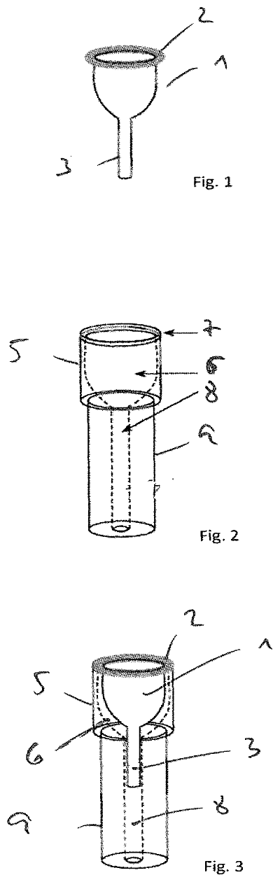

FIG. 2 a schematic representation of a mounting bell and a connection unit;

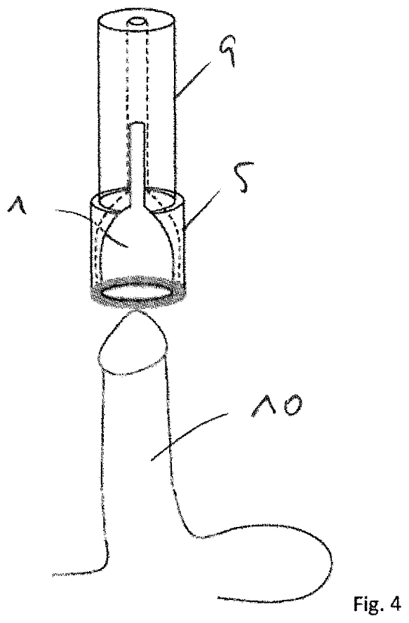

FIG. 3 an illustration according to FIG. 2 with an inserted urinal condom;

FIG. 4 a representation of the application process of the device according to the invention for applying the condom for use as intended;

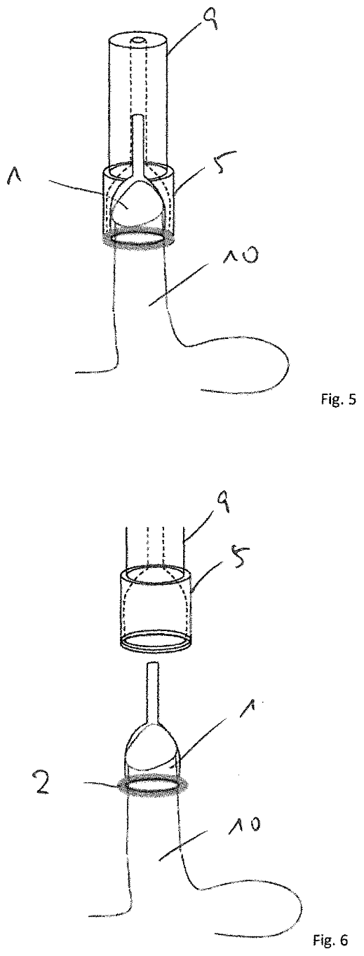

FIG. 5 a representation according to FIG. 4 in a continued position;

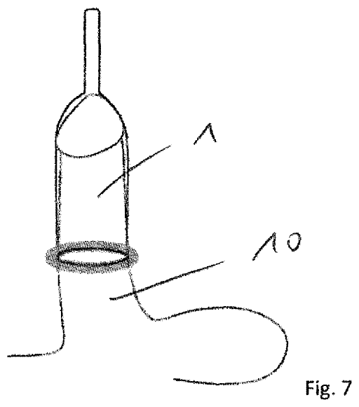

FIG. 6 a representation according to FIG. 4 in a final position;

FIG. 7 an illustration of the urinal condom in its intended position;

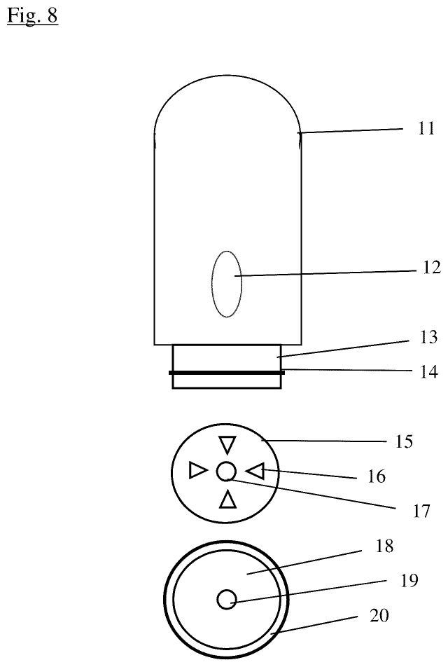

FIG. 8 an illustration of the connection surfaces between the vacuum pump and the connection unit, and

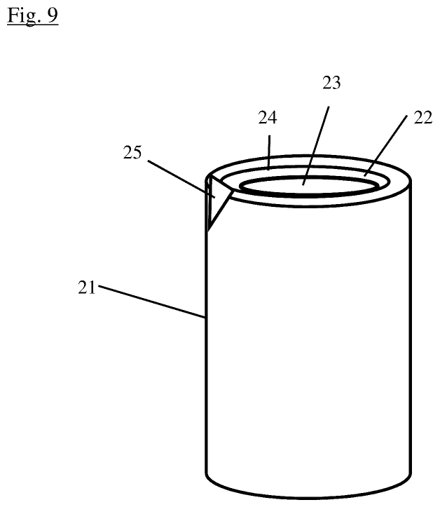

FIG. 9 an illustration of the upper receiving area of a receiving bell.

FIG. 1 shows a typical urinal condom 1, which is made of an elastic material and forms a sheath that is rolled up into a ring. There is a tube attachment at the front end of the urinal condom.

The winding 2 serves to simplify the application of the condom, the hose attachment 3 serves to remove it. FIGS. 2 and 3 show an example of a device according to the invention.

A receiving bell 5 has an inner bell-shaped cavity 6. This preferably has a shape that corresponds to the intended size of a urinal condom. A groove 7 running around the upper edge serves to accommodate the wound-up area of the urinal condom, the so-called condom ring. The inside of the receiving bell 5 is fluidically connected to a tubular channel 8 of the connecting unit 9. The receiving bell 5 and the connecting unit 9 are cylindrical on the outside. A flow-tight connection between the receiving bell 5 and the connection unit 9 can be produced in any conventional way, by pressing, screwing, using sealing rings and the like.

As FIG. 3 in particular shows, a urinal condom can be inserted into the device in such a way that the uncoiled area is accommodated in the receiving bell and the tube attachment 3 is inserted into the tubular channel.

The application is shown in FIGS. 4 to 6. The connection unit connected to a device for generating a vacuum, called a vacuum pump, which is not shown, ensures that when the device is placed on a penis 10, the urinal condom 1 allows suction after being placed on the tip while supporting itself on the receiving bell. The vacuum can be generated manually or electrically. After the penis 10 has been sucked into the urinal condom 1, as shown in FIG. 5, the device can be removed. A correspondingly higher static friction causes the condom to be removed from the receiving bell. This can be assisted by switching off the negative pressure.

As shown in FIG. 7, it is now only necessary to unroll the rolled-up condom area as usual.

FIG. 8 shows the interaction of the elements according to the invention. In the example shown, the vacuum pump 11 is shown as an electric pump. This has an operating unit 12, which can be used to switch the pump on and off. At one end, the pump has a connection piece 13, which has a circumferential sealing ring 14 in the example shown. The bottom view of the lower surface 15 shows the suction channel 17 as the central opening. Protrusions 16 are formed on the surface 15. These can be flat and their sole function is to prevent direct suction of the adjacent surfaces.

The surface from 18 of the connection unit comes into contact with the surface 15. This has the opening 19, which represents the end of the suction channel of the connection unit. In the design example shown, the surface 18 is surrounded by a sealing edge 20.

The connecting piece 13 of the pump is inserted into the tube formed by the sealing ring 20 until the surface 15 comes to rest on the surface 18. They are only spaced apart by the spacer elements 16. The inner wall of the sealing edge 20 interacts with the sealing ring 14 so that the vacuum generated by the vacuum pump 11 is transferred to the suction channel of the connection unit via the opening 19.

FIG. 9 shows the receiving area of a receiving bell 21. Surrounding the air channel 23, a circumferential phase or groove 22 is formed on the inner upper edge, whereby a receiving area 24 is formed for the wound-up area of the urinal condom, the so-called condom ring. This condom ring protrudes slightly beyond the upper edge of the receiving bell 21. The gripping groove 25, a kind of recess or depression in the area of the outer upper edge of the receiving bell 21, makes the condom ring easier to reach and grasp. A large number of gripping grooves can be formed in the area of the upper edge.

The embodiment example described is for explanatory purposes only and is not restrictive.

### REFERENCE SYMBOL

1 Urinal condom
2 Winding
3 Tube attachment
5 Receiving bell
6 Cavity
7 Groove
8 Tubular channel
9 Connecting unit
10 Penis
11 Vacuum pump
12 Control unit
13 Connection piece 14 Sealing ring
15 Lower surface connection piece
16 Spacer elements
17 Suction duct 18 Connection point connection unit
19 Suction channel
20 Sealing rim
21 Recording bell
22 Groove
23 Channel
24 Recording area
25 Gripping groove

1. System for applying a urinal condom (1) for use as intended, having a receiving bell (5), a vacuum pump and a connecting unit (9) for fluidically connecting the receiving bell (5) to the vacuum pump, the connecting unit (9) having a tubular channel (8) to which the receiving bell (5) is fluidically connected, the tubular channel (8) being designed on the receiving bell side as a receptacle for a hose attachment of the urinal condom, characterized in that the connecting unit has, on the side to be connected to the vacuum pump, a surface which is congruent with a surface formed on the vacuum pump, at least one of the two surfaces having an uneven structure, and the vacuum pump and the connecting unit being fluidically connected via the two superimposed surfaces.

2. System according to claim 1, wherein the two surfaces lying on top of each other are sealed at the edges.

3. System according to claim 2, wherein one of the two surfaces of the connection unit or the vacuum pump is surrounded by a sealing edge into which the respective other element can be inserted in a flow-tight manner.

4. System according to claim 1, wherein a seal is inserted between the sealing edge and the surface interacting therewith.

5. System according to claim 1, wherein the receiving bell (5) has a circumferential groove (7) on an upper edge.

6. System according to claim 5, wherein the receiving bell has gripping grooves on the outside in the area of the upper edge.

7. System according to claim 1, wherein the receiving bell (5) is cylindrical on the outside.

8. System according to claim 1, wherein the receiving bell (5) is detachably connected to the connecting unit (9).

9. System according to claim 1, wherein the connecting unit (9) is essentially cylindrical.

10. System according to claim 1, wherein the receiving bell (5) and the connecting unit (9) are made of plastic.
